(19) United States
(12) Patent Application Publication
(10) Pub. No.: US 2011/0223603 A1
(43) Pub. Date: Sep. 15, 2011

(54) DIAGNOSING AND MONITORING RESPONSE TO TREATMENT OF NON-INCONTINENT UROLOGICAL AND RELATED DISEASES

(75) Inventors: Martin Heath Bluth, Southfield, MI (US); Wellman Cheung, Briarwood, NY (US)

(73) Assignees: Martin Heath Bluth, Southfield, MI (US); Wellman Cheung, Briarwood, NY (US)

(21) Appl. No.: 13/130,362

(22) PCT Filed: Nov. 20, 2009
(86) PCT No.: PCT/US09/65383

§ 371 Date: May 20, 2011

Related U.S. Application Data
(60) Provisional application No. 61/116,777, filed on Nov. 21, 2008.
(60) Provisional application No. 61/116,796, filed on Nov. 21, 2008.
(60) Provisional application No. 61/116,794, filed on Nov. 21, 2008.
(60) Provisional application No. 61/116,791, filed on Nov. 21, 2008.

(51) Int. Cl. C12Q 1/68; C12Q 1/02

(52) U.S. Cl. 435/6.11; 435/29

(57) ABSTRACT

Techniques for diagnosing and monitoring response to treatment of non-incontinent urological disorders (NIUD) in a patient are provided. For example, a technique for diagnosing NIUD in a patient includes obtaining peripheral blood-derived nucleic acid containing nucleated acellular components such as serum and/or plasma and/or nucleated cellular components from the patient to provide a reporter function in the patient. Also, a technique for monitoring response to treatment of NIUD in a patient includes obtaining peripheral blood-derived nucleic acid containing nucleated acellular components such as serum and/or plasma and/or nucleated cellular components from the patient to provide a reporter function in the patient.

### CROSS-REFERENCE TO RELATED APPLICATION(S)

This application claims the benefit of U.S. provisional application Ser. No. 61/116,777, filed on Nov. 21, 2008, U.S. provisional application Ser. No. 61/116,791, filed on Nov. 21, 2008, U.S. provisional application Ser. No. 61/116,794, filed on Nov. 21, 2008, and U.S. provisional application Ser. No. 61/116,796, filed on Nov. 21, 2008, the disclosures of which are incorporated by reference herein in their entireties for all purposes.

### FIELD OF THE INVENTION

The present invention relates generally to immunology, and more particularly relates to diagnosing and monitoring response to treatment of one or more diseases.

### BACKGROUND OF THE INVENTION

Non-incontinent urological disorders (NIUD) affect a majority of the male and female population. Conditions herein referred to as NIUD are often treatable, but many afflicted patients remain undiagnosed or untreated, due, in large part, to the invasive existing diagnostic approaches. Furthermore, they can present in patients with other diseases making the correct diagnosis in these patients very difficult.

NIUD continue to present diagnostic challenges, and confirmation of disease often requires invasive procedures. For example, NIUD can be diagnosed by history and/or confirmed by invasive biopsy, which represents an invasive approach. Biopsies require appropriate tissue representation and can be misdiagnosed in the event of improper sample procurement. Additionally, biopsies carry morbidity risks and are therefore undesirable.

### SUMMARY OF THE INVENTION

The invention, in illustrative embodiments thereof, provides techniques for diagnosing non-incontinent urological disorders (NIUD) and/or monitoring response to treatment of NIUD.

In accordance with one aspect of the invention, a technique for diagnosing NIUD in a patient includes obtaining blood-derived nucleic acid containing nucleated acellular components such as serum and/or plasma, and/or nucleated cellular components such as leukocytes which include polymorphonuclear (PMN) cells, peripheral blood mononuclear cells (PBMC), basophils, eosinophils in addition to reticulocytes and/or platelets from the patient to provide a reporter function in the patient.

Additionally, in accordance with another aspect of the invention, a technique for monitoring response to treatment of NIUD in a patient includes obtaining peripheral blood-derived nucleic acid containing nucleated acellular components such as serum and/or plasma and/or nucleated cellular components such as leukocytes which include polymorphonuclear (PMN) cells, peripheral blood mononuclear cells (PBMC), basophils, eosinophils in addition to reticulocytes and/or platelets from the patient to provide a reporter function in the patient.

These and other features, objects and advantages of the present invention will become apparent from the following detailed description of illustrative embodiments thereof, which is to be read in connection with the accompanying drawings.

### BRIEF DESCRIPTION OF THE DRAWINGS

FIG. 1 is a flow diagram illustrating an exemplary technique for diagnosing NIUD in a patient, according to an embodiment of the present invention;

FIG. 2 is a flow diagram illustrating an exemplary technique for monitoring response to treatment of NIUD in a patient, according to an embodiment of the present invention; and

FIG. 3 is a flow diagram illustrating an exemplary technique for prognosticating NIUD in a patient, according to an embodiment of the present invention.

### DETAILED DESCRIPTION OF PREFERRED EMBODIMENTS

Initially, the complete disclosures of U.S. provisional patent application Ser. No. 61/116,777 to Bluth et al. entitled “Monitoring Response to Treatment of Urological Disease,” filed on Nov. 21, 2008, U.S. provisional patent application Ser. No. 61/116,791 to Bluth et al. entitled “Diagnosing and Monitoring Response to Treatment of Pancreatic Disease,” filed on Nov. 21, 2008, U.S. provisional patent application Ser. No. 61/116,794 to Bluth et al. entitled “Diagnosing and Monitoring Response to Treatment of Benign Prostatic Hypertrophy,” filed on Nov. 21, 2008, and U.S. provisional patent application Ser. No. 61/116,796 to Bluth et al. entitled “Diagnosing and Monitoring Response to Treatment of Erectile Dysfunction,” filed on Nov. 21, 2008, are expressly incorporated herein by reference in their entireties for all purposes.

NIUD, as noted above, have traditionally presented diagnostic difficulties, and confirmation of disease often requires invasive approaches. As such, a blood-based assay with appropriate diagnostic sensitivity and specificity would complement or obviate these invasive testing procedures and would therefore be advantageous.

With respect to one or more embodiments of the invention, non-malignant disorders, referred to herein generally as NIUD, can include, but are not limited to, erectile dysfunction (ED), interstitial cystitis, benign prostatic hypertrophy (BPH), bladder outlet obstruction, chronic pelvic pain, neurogenic bladder, chronic prostatitis, urethral syndrome, erectile dysfunction, male infertility, female infertility, orgasmic disorder, ovarian cyst, renal cyst, male sexual dysfunction, female sexual dysfunction, synthetic foreign body (i.e. stent) placement, urinary lithiasis, benign disease, cystic renal disease, angiomyolipoma, renal oncocytoma, adrenal adenoma, fecal incontinence, irritable bowel syndrome, premature ejaculation, pelvic organ prolepses, chronic constipation, chronic diarrhea, and detrussor sphincter dyssynergia.

As described herein, peripheral blood-derived nucleic acid containing nucleated acellular components, such as, for example, serum and/or plasma and/or nucleated cellular components such as leukocytes which include polymorphonuclear (PMN) cells, peripheral blood mononuclear cells (PBMC), basophils, eosinophils in addition to reticulocytes and/or platelets, can provide a reporter function in solid organ retroperitoneal disease. PMN cells are also referred to as granulocytes and PBMC include mononucleated cells consisting of B cells, T cells, monocytes, and natural killer cells, among others. As such, as described herein, one or more embodiments of the invention use peripheral blood-derived nucleic acid containing nucleated acellular components such as serum and/or plasma and/or nucleated cellular components such as leukocytes which include PMN cells, PBMC, basophils, eosinophils in addition to reticulocytes and/or platelets as a marker for patients with NIUD and the use peripheral blood-derived nucleic acid containing nucleated acellular components such as serum and/or plasma and/or nucleated cellular components such as leukocytes which include PMN cells, PBMC, basophils, eosinophils in addition to reticulocytes and/or platelets as a marker for patients with NIUD.

The term “patient” as used herein is intended to refer broadly to mammalian subjects, and more preferably refers to humans receiving medical attention (e.g., diagnosis, monitoring, etc.), care or treatment.

With respect to one or more embodiments of the invention, proof of concept is derived from similar studies utilizing said technology applied to urological disease namely overactive bladder (OAB). Similar to OAB, NIUD represents another urological disease where the afflicted organs can be intracavitary, composed of solid tissue and is not easily accessible. The following data derived from OAB can easily be derived for NIUD by one skilled in the art given the teachings herein. For example, from OAB, one should be able to derive adaptation of the techniques detailed herein to NIUD as these, like OAB, represent diseases of the urological spectrum. This is in contrast to diseases which have underlying pathologies which may be unrelated to immune related responses. For example, stroke would be considered to result from a coagulopathy which would form a clot that subsequently lodges in the small vessels of the brain.

By way of example, one or more embodiments of the invention find the same gene product (for example, PDGF-C) in PMN cells (cellular) and serum/plasma (acellular) components. By way of a nexus, PBMC are comprised of many types of immune cells such as B cells, T cells, monocytes etc. As such, PMN cells, platelets and reticulocytes represent other types of immune cells or related cells which can provide additional reporter function. Further, serum and plasma can include the nucleic acid such as RNA normally contained within the nucleated cells (PBMC, PMN, etc.) and, as such, can be interrogated directly to provide a reporter function of immune or immune-related cells without the need for obtaining the immune cells directly.

Twenty-one patients were assessed (as confirmed by urodynamics) for OAB and structural integrity (for example, pelvic prolapse, bladder neck dysfunction, bladder capacity, stress incontinence, and involuntary bladder contraction). Patients with a history of recent surgery, infection, or positive valsalva leak point pressure or negative for involuntary bladder contraction, as demonstrated by urodynamics, were excluded. Whole blood was obtained from patients and appropriately matched controls (n=6) and peripheral blood mononuclear cells (PBMC) were separated by density centrifugation (Ficoll-Paque). In one or more embodiments of the invention, for example, between one and six mL of whole blood can be collected.

Ribonucleic acid (RNA) was isolated from PBMC and converted to complementary (cRNA) and subjected to microarray analysis (human U133A 2.0 gene chip). Data are expressed as fold change and were analyzed using Lima Package from Bioconductor. Genes with greater than two-fold change were considered positive.

Changes between patients with OAB were compared with healthy controls as a total, and we also separated these comparisons into OAB versus controls for men only and women only. The analysis can have gender-based importance as some genes which are hormonally-linked behaved differently in each gender. For example, PGRMC1 (progesterone receptor membrane component 1) was found to be downregulated in females with OAB but not in males, suggesting that sex-based analysis will provide additional genes of interest which can serve to diagnose or monitor treatment response to OAB in females as compared to males.

The average patient age was 51 years (range=31 to 73 years of age) and were predominantly female (76%). The majority of patients had involuntary bladder contraction (62%), and stress incontinence (52%), and most female patients had pelvic prolapse (69%). Forty-three percent of patients had bladder neck dysfunction and all patients had normal to low bladder capacity. Microarray analysis revealed that 16 genes were differentially regulated (8 upregulated and 8 downregulated) in all patients with OAB compared with controls. By way of example, PGRMC1 (progesterone receptor membrane component 1), EIF2S3 (eukaryotic initiation factor), C3AR1 (complement receptor) and 3 unknown genes were down-regulated. Upregulated genes included, for example, MYOM2 (myomesin M-protein), a cytoskelatal protein involved in structural integrity.

Gender-based analysis demonstrated 74 genes differentially regulated in males (25 upregulated and 49 downregulated), and 30 in females (13 upregulated and 17 downregulated). Of these, platelet-derived growth factor (PDGF), MFAP3L (a microfibrillar-associated protein), and TPM1 (tropomyosin) were downregulated in all sets analyzed. PDGF, MFAP3L and TPM1, as is well-known in the art, have structurally-related importance as a result of similarity to structural function.

Microarray analysis revealed many genes which were differentially regulated in PBMC from OAB patients. For example, OAB patients, including those with involuntary detrusor contractions, exhibited differential expression of regulatory/structural genes when compared with healthy controls. To this end, PDGF, MFAP and TPM1, as illustrated herein, may be important in regulating structural integrity of bladder and supporting tissues. These data suggest that PBMC provides a reporter function for patients with urological disease such as OAB and can serve as a diagnostic marker and/or monitor response to therapy.

With the ability to correlate solid tissue and blood-based gene changes, PBMC-derived gene screens can mirror diseased solid tissue gene changes. The downregulation of PDGF and tropomyosisn (which is involved in smooth muscle contraction and maintaining bladder integrity) in all data sets (males, females and combined analysis) further highlights the involvement of PBMC-derived gene screens in the pathogenesis of urological disease. Also, the upregulation of Adrenomedullin (ADM) (which is expressed in bladder smooth muscle among other organs) in all data sets may be part of a compensatory feedback loop in response to low bladder muscle tone or tissue integrity.

In one or more embodiments of the invention, the use of PBMC-derived microarray gene changes can also be censored to highlight gender-specific responses. Such techniques can further elucidate sex-specific mechanisms and provide novel therapeutic possibilities. As detailed herein, analysis of male data sets revealed downregulation of caldesmon (CALD1) (which is involved in detrusor smooth muscle integrity and bladder remodeling). Also, synuclein (SNCA), which has been associated with Parkinson's disease and dementia, was downregulated in the male data set, signaling that SNCA may be involved in maintaining neurologic tone of the bladder and its decrease may contribute to the severity of urological disease, particularly in males. The downregulation of progesterone receptor membrane component 1 (PGRMC1) in the female data set can provide a gender specific marker as well, as progesterone receptors have been associated with bladder integrity.

As described herein, one or more embodiments of the present invention include a non-invasive, safe method of providing biomarkers (that is, substances used as indicators of one or more biologic states) for NIUD when compared with biopsy of solid organ. A biomarker can be, for example, a substance whose detection indicates a particular disease state. More specifically, a biomarker can indicate a change in expression or state of a protein that correlates with the risk or progression of a disease, or with the susceptibility of the disease to a given treatment.

The techniques described herein facilitate identifying genes (for example, previously unknown and/or unanticipated genes) involved in NIUD and other urological disease, as well as benefiting drug design and development. Additionally, one or more embodiments of the present invention highlight differentially expressed genes with respect to disease severity and/or in response to drug treatment over time which can provide mechanisms of drug action and further understanding of disease pathophysiology.

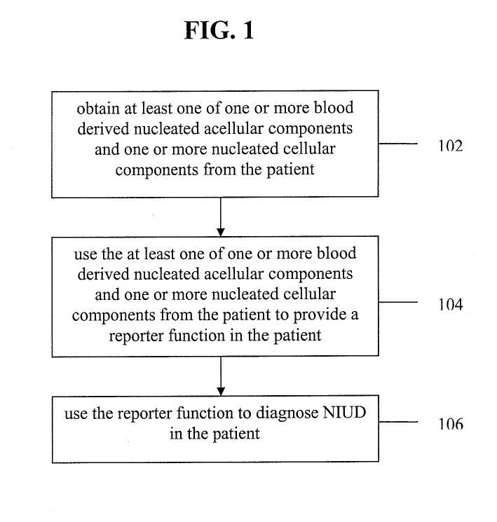

FIG. 1 is a flow diagram illustrating an exemplary methodology for diagnosing NIUD in a patient, according to an embodiment of the present invention. In step 102, at least one of one or more blood derived nucleated acellular components and one or more nucleated cellular components is obtained from the patient. Step 104 includes using the at least one of one or more blood derived nucleated acellular components and one or more nucleated cellular components obtained from the patient for providing a reporter function in the patient. In step 106, the reporter function is used for diagnosing NIUD in the patient.

In one or more embodiments of the invention, the patient includes a patient devoid of an immunomodulatory condition and immunomodulatory therapy. The diseases detailed herein are not generally thought to be of an immunological nature versus inflammatory disease. As such, the selected patients are preferably those which do not have immunomodulatory conditions or immunomodulatory therapy. This allows for biomarker discovery by interrogating the immune or immune related system in a relatively pure disease state without confounding immunomodulatory activity.

Obtaining peripheral blood-derived nucleic acid containing nucleated acellular components such as serum and/or plasma and/or nucleated cellular components, such as, for example, leukocytes which include polymorphonuclear (PMN) cells, peripheral blood mononuclear cells (PBMC), basophils, eosinophils in addition to reticulocytes and/or platelets from a patient, can include obtaining whole blood from a patient (for example, in an amount in a range of about one to six milliliters (ml)), and isolating peripheral blood derived nucleic acid containing nucleated acellular components such as serum and/or plasma and/or nucleated cellular components such as leukocytes which include PMN cells, PBMC, basophils, eosinophils in addition to reticulocytes and/or platelets from the whole blood.

Providing a reporter function in the patient may include, but is not limited to, processing the peripheral blood derived nucleic acid containing nucleated acellular components such as serum and/or plasma and/or nucleated cellular components such as leukocytes which include PMN cells, PBMC, basophils, eosinophils in addition to reticulocytes and/or platelets to isolate ribonucleic acid (RNA) for analysis. Such analysis includes comparing gene changes in the patient versus gene changes in a control sample to provide a screening modality to ascertain one or more genes involved in a pathogenesis of NIUD.

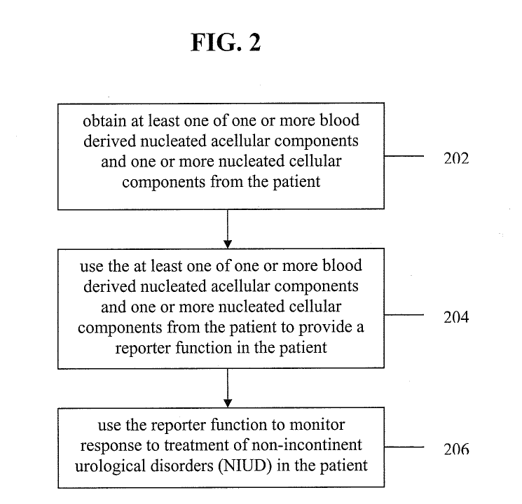

FIG. 2 is a flow diagram illustrating an exemplary methodology for monitoring disease severity and/or response to treatment of NIUD in a patient, according to an embodiment of the present invention. As apparent from the figure, step 202 includes obtaining at least one of one or more blood derived nucleated acellular components and one or more nucleated cellular components from the patient. Step 204 includes using the at least one of one or more blood derived nucleated acellular components and one or more nucleated cellular components from the patient to provide a reporter function in the patient. Step 206 includes using the reporter function to monitor response to treatment of NIUD in the patient. In one or more embodiments of the invention, the patient includes a patient devoid of an immunomodulatory condition and immunomodulatory therapy.

Obtaining peripheral blood derived nucleic acid containing nucleated acellular components such as serum and/or plasma and/or nucleated cellular components such as leukocytes which include PMN cells, PBMC, basophils, eosinophils in addition to reticulocytes and/or platelets from a patient may include obtaining whole blood from a patient (for example, in an amount in a range of about one to six milliliters), and isolating peripheral blood derived nucleic acid containing nucleated acellular components such as serum and/or plasma and/or nucleated cellular components such as leukocytes which include PMN cells, PBMC, basophils, eosinophils in addition to reticulocytes and/or platelets from the whole blood.

Providing a reporter function in the patient can include, for example, processing the peripheral blood derived nucleic acid containing nucleated acellular components such as serum and/or plasma and/or nucleated cellular components such as leukocytes which include PMN cells, PBMC, basophils, eosinophils in addition to reticulocytes and/or platelets to isolate RNA for analysis, wherein the analysis may include comparing gene changes in the patient versus gene changes in a control sample to provide a screening modality to ascertain one or more genes involved in a pathogenesis of NIUD. Providing a reporter function can also include ascertaining one or more gene changes pre- and post-treatment of urological disease in the patient. Further, ascertaining gene changes pre- and post-treatment of urological disease in the patient can include elucidating a therapeutic mechanism (for example, a drug mechanism) of action.

As noted above, comparing gene changes in a patient versus gene changes in a control samples provides a screening modality to mine for unique genes potentially involved in the pathogenesis of NIUD, which can be subsequently confirmed using conventional methods such as, for example, western blot, enzyme linked immunosorbant assay, polymerase chain reaction, etc. Principles of the present invention determine any gene changes that occur pre- and post-therapy, thereby providing a putative mechanism of action for the therapeutic treatment of NIUD.

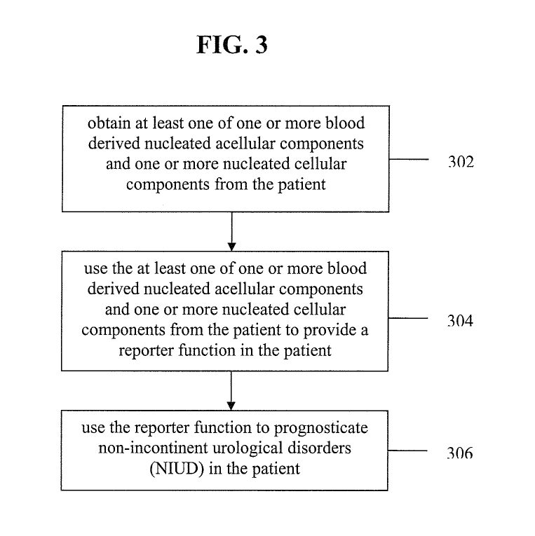

FIG. 3 is a flow diagram illustrating an exemplary methodology for prognosticating NIUD in a patient, according to an embodiment of the present invention. Step 302 includes obtaining at least one of one or more blood derived nucleated acellular components and one or more nucleated cellular components from the patient. Step 304 includes using the at least one of one or more blood derived nucleated acellular components and one or more nucleated cellular components from the patient to provide a reporter function in the patient. Step 306 includes using the reporter function to prognosticate NIUD in the patient.

In one or more embodiments of the invention, the patient includes a patient devoid of an immunomodulatory condition and immunomodulatory therapy. The blood derived nucleated acellular components can include, for example, serum and/or plasma, and the blood derived nucleated cellular components can include PMN cells, PBMC, platelets, eosinophils, basophils and/or reticulocytes.

Obtaining blood-derived nucleated acellular components and/or nucleated cellular components from the patient can include obtaining whole blood from the patient, and isolating one or more blood derived nucleated acellular components and/or one or more nucleated cellular components from the whole blood. Also, providing a reporter function in the patient can include processing the one or more blood derived nucleated acellular components and/or one or more nucleated cellular components to isolate ribonucleic acid (RNA) for analysis, wherein the analysis includes comparing one or more gene changes in the patient versus one or more gene changes in known disease state samples to provide a screening modality to ascertain severity of the NIUD.

Although illustrative embodiments of the present invention have been described herein, it is to be understood that the invention is not limited to those precise embodiments, and that various other changes and modifications may be made by one skilled in the art without departing from the scope or spirit of the invention.

1. A method for diagnosing non-incontinent urological disorders (NIUD) in a patient, the method comprising the steps of:
obtaining at least one of one or more blood derived nucleated acellular components and one or more nucleated cellular components from the patient;
using the at least one of the one or more blood-derived nucleated acellular components and the one or more nucleated cellular components obtained from the patient for providing a reporter function in the patient; and
using the reporter function to diagnose NIUD in the patient.

2. The method of claim 1, wherein the one or more blood derived nucleated acellular components comprise at least one of serum and plasma.

3. The method of claim 1, wherein NIUD comprises at least one of erectile dysfunction, interstitial cystitis, benign prostatic hypertrophy, bladder outlet obstruction, chronic pelvic pain, neurogenic bladder, chronic prostatitis, urethral syndrome, erectile dysfunction, male infertility, female infertility, orgasmic disorder, ovarian cyst, renal cyst, male sexual dysfunction, female sexual dysfunction, synthetic foreign body (i.e. stent) placement, urinary lithiasis, benign disease, cystic renal disease, angiomyolipoma, renal oncocytoma, adrenal adenoma, fecal incontinence, irritable bowel syndrome, premature ejaculation, pelvic organ prolepses, chronic constipation, chronic diarrhea, and detrussor sphincter dyssynergia.

4. The method of claim 1, wherein the patient comprises a patient devoid of an immunomodulatory condition and immunomodulatory therapy.

5. The method of claim 1, wherein the one or more blood-derived nucleated cellular components comprise at least one of polymorphonuclear (PMN) cells, peripheral blood mononuclear cells (PBMC), platelets, eosinophils, basophils and reticulocytes.

6. The method of claim 1, wherein the step of obtaining at least one of one or more blood-derived nucleated acellular components and one or more nucleated cellular components from the patient comprises:
obtaining whole blood from the patient; and
isolating at least one of one or more blood derived nucleated acellular components and one or more nucleated cellular components from the whole blood.

7. (canceled)

8. The method of claim 1, wherein providing a reporter function in the patient comprises processing the at least one of one or more blood derived nucleated acellular components and one or more nucleated cellular components to isolate ribonucleic acid (RNA) for analysis, wherein the analysis comprises comparing one or more gene changes in the patient versus one or more gene changes in a control sample to provide a screening modality to ascertain one or more genes involved in a pathogenesis of NIUD.

9. A method for monitoring response to treatment of non-incontinent urological disorders (NIUD) in a patient, the method comprising the steps of:
obtaining at least one of one or more blood derived nucleated acellular components and one or more nucleated cellular components from the patient;
using the at least one of one or more blood derived nucleated acellular components and one or more nucleated cellular components obtained from the patient for providing a reporter function in the patient; and
using the reporter function to monitor response to treatment of NIUD in the patient.

10. The method of claim 9, wherein the patient comprises a patient devoid of an immunomodulatory condition and immunomodulatory therapy.

11. The method of claim 9, wherein the one or more blood-derived nucleated acellular components comprise at least one of serum and plasma.

12. The method of claim 9, wherein NIUD comprises at least one of overactive bladder, erectile dysfunction, interstitial cystitis, benign prostatic hypertrophy, bladder outlet obstruction, chronic pelvic pain, neurogenic bladder, chronic prostatitis, urethral syndrome, erectile dysfunction, male infertility, female infertility, orgasmic disorder, ovarian cyst, renal cyst, male sexual dysfunction, female sexual dysfunction, synthetic foreign body (i.e. stent) placement, urinary lithiasis, benign disease, cystic renal disease, angiomyolipoma, renal oncocytoma, adrenal adenoma, fecal incontinence, irritable bowel syndrome, premature ejaculation, pelvic organ prolepses, chronic constipation, chronic diarrhea, and detrussor sphincter dyssynergia.

13. The method of claim 9, wherein the one or more blood-derived nucleated cellular components comprise at least one of polymorphonuclear (PMN) cells, peripheral blood mononuclear cells (PBMC), platelets, eosinophils, basophils and reticulocytes.

14. The method of claim 9, wherein the step of obtaining at least one of one or more blood-derived nucleated acellular components and one or more nucleated cellular components from the patient comprises:
obtaining whole blood from the patient; and
isolating at least one of one or more blood derived nucleated acellular components and one or more nucleated cellular components from the whole blood.

15. (canceled)

16. The method of claim 9, wherein providing a reporter function in the patient comprises processing the at least one of one or more blood-derived nucleated acellular components and one or more nucleated cellular components to isolate ribonucleic acid (RNA) for analysis, wherein the analysis comprises comparing one or more gene changes in the patient versus one or more gene changes in a control sample to provide a screening modality to ascertain one or more genes involved in a pathogenesis of NIUD.

17. The method of claim 9, wherein providing a reporter function in the patient comprises ascertaining one or more gene changes pre- and post-treatment of NIUD in the patient.

18. The method of claim 17, wherein ascertaining one or more gene changes pre- and post-treatment of NIUD in the patient further comprises elucidating a therapeutic mechanism of action.

19. A method for prognosticating non-incontinent urological disorders (NIUD) in a patient, the method comprising:
obtaining at least one of one or more blood-derived nucleated acellular components and one or more nucleated cellular components from the patient;
using the at least one of one or more blood-derived nucleated acellular components and one or more nucleated cellular components from the patient for providing a reporter function in the patient; and
using the reporter function to prognosticate NIUD in the patient.

20. The method of claim 19, wherein the patient comprises a patient devoid of an immunomodulatory condition and immunomodulatory therapy.

21. The method of claim 19, wherein the one or more blood-derived nucleated acellular components comprise at least one of serum and plasma, and the one or more blood-derived nucleated cellular components comprise at least one of polymorphonuclear (PMN) cells, peripheral blood mononuclear cells (PBMC), platelets, eosinophils, basophils and reticulocytes.

22. (canceled)

23. The method of claim 19, wherein providing a reporter function in the patient comprises processing the at least one of one or more blood-derived nucleated acellular components and one or more nucleated cellular components to isolate ribonucleic acid (RNA) for analysis, wherein the analysis comprises comparing one or more gene changes in the patient versus one or more gene changes in known disease state samples to provide a screening modality to ascertain severity of a non-incontinent urological disorder.
